(19) United States
(12) Patent Application Publication
(10) Pub. No.: US 2026/0125639 A1
(43) Pub. Date: May 7, 2026

(54) THREE-DIMENSIONAL CO-CULTURE SYSTEM FOR EMBRYOS CULTURED IN VITRO AND USES THEREOF

(71) Applicant: Colossal Biosciences Inc., Austin, TX (US)

(72) Inventors: Leyi LI, Austin, TX (US); Eriona HYSOLLI, Austin, TX (US); Cassdiy BEARD, Austin, TX (US); Jie OUYANG, Austin, TX (US)

(21) Appl. No.: 19/119,976

(22) PCT Filed: Oct. 10, 2023
(86) PCT No.: PCT/US2023/076422

§ 371 Date: Apr. 10, 2025

Related U.S. Application Data
(60) Provisional application No. 63/378,929, filed on Oct. 10, 2022.

(51) Int. Cl. C12N 5/073

(52) U.S. Cl. CPC C12N 5/0604; C12N 2501/392; C12N 2502/243; C12N 2513/00; C12N 2533/54

(57) ABSTRACT

Provided herein are three-dimensional co-culture systems, methods of using three-dimensional co-culture systems, and kits comprising three-dimensional co-culture systems.

### CROSS REFERENCE TO RELATED APPLICATION

This application claims priority to U.S. Provisional Application No. 63/378,929, filed on Oct. 10, 2022, the disclosure of which is incorporated herein by reference in its entirety.

### FIELD OF THE INVENTION

This invention relates to the field of biotechnology. Provided herein are three-dimensional co-culture systems for culturing embryos in vitro. The three-dimensional co-culture systems comprise (a) a three-dimensional structure, (b) at least one somatic cell; (c) at least one embryo; and (d) cell culture media.

### BACKGROUND OF THE INVENTION

In vitro production of embryos has several advantages over in vivo-derived embryo production, including, but not limited to the efficient selection of superior genetics for the transfer of genetic modification to rapidly obtain animals with desirable traits. Genetic engineering can provide a powerful tool to aid in the understanding of basic mechanisms regulating physiology.

As the in vitro environment is still suboptimal for embryo growth and development, there is a need to develop a better system for culturing embryos in vitro to mimic the in vivo environment to provide robust and viable embryos for a higher success rate for in vitro fertilization procedures, better efficiency in reproductive technologies in agriculturally important animals, and better efficiency for the conservation of extinct and endangered species through reproductive technologies.

### BRIEF SUMMARY OF THE INVENTION

In one general aspect, the invention relates to a three-dimensional co-culture system. The three-dimensional co-culture system can, for example, comprise (a) a three-dimensional structure; (b) at least one somatic cell; (c) at least one embryo; and (d) cell culture media.

Also provided are methods of growing an embryo in a three-dimensional co-culture system. The methods comprise (a) culturing at least one somatic cell; (b) embedding the at least one somatic cell in a three-dimensional structure; (c) obtaining at least one embryo; and (d) embedding or placing the at least one embryo in or near the three-dimensional structure; wherein the at least one somatic cell and at least one embryo are grown in or near the three-dimensional structure in cell culture media.

Also provided are kits comprising (a) a three-dimensional structure; (b) at least one somatic cell; (c) at least one embryo; and (d) cell culture media.

In certain embodiments, the three-dimensional structure is a scaffold-based structure. The scaffold-based structure can, for example, comprise a gel-like material or a structural scaffold. The gel-like material can, for example, be selected from a hydrogel, an agarose, a basement membrane extract, or an extracellular matrix. In certain embodiments, the scaffold-based structure is created by a three-dimensional printer.

In certain embodiments, the three-dimensional structure is a scaffold-free structure. The scaffold-free structure can, for example, comprise cell aggregates forming a three-dimensional structure.

In certain embodiments, the somatic cell is selected from a skin cell, a bone cell, a blood cell, a connective tissue cell, a cumulus cell, a granulosa cell, and/or a cell from the reproductive tract.

In certain embodiments, the embryo is selected from the group consisting of a mammalian embryo, an avian embryo, a reptilian embryo, a fish embryo, an amphibian embryo, and a marsupial embryo. The embryo can, for example, be a mammalian embryo.

In certain embodiments, the cell culture media is selected from at least one of minimum essential media (MEM), Dulbecco's modified eagle medium (DMEM), Roswell Park Memorial Institute medium (RPMI), Dulbecco's modified eagle medium/nutrient mixture F-12 (DMEM/F-12), N2B27, M16, potassium supplemented SOM (KSOM), tissue culture medium-199 (TCM-199), or a custom-made medium. The cell culture media can, for example, comprise at least one of a sodium ion (Na⁺), a potassium ion (K⁺), a calcium ion (Ca²⁺), or a magnesium ion (Mg²⁺).

In certain embodiments, the somatic cell is embedded in or placed near the three-dimensional structure. In certain embodiments, the embryo is embedded in or placed near the three-dimensional structure.

In certain embodiments, the three-dimensional co-culture system further comprises a bioreactor.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A and 1B show representative images of a monolayer cell culture (FIG. 1A) and a three-dimensional (3D) cell culture (FIG. 1B).

FIG. 2 shows representative images demonstrating the progress of a three-dimensional cell culture over the course of 8 days. The somatic cells within the three-dimensional culture proliferated and connected to each other to form a three-dimensional structure of cell aggregate. Over the course of the 8-day culture, more structures were formed.

FIG. 3 shows a representative image of a three-dimensional co-culture system to culture embryos in vitro. At least one embryo was embedded into the three-dimensional structure of somatic cells, which provided favorable growth conditions for the embryo. The three-dimensional structure also provided physical support to maintain the spatial morphological shape of the embryo during the growth and the development of the embryo.

FIG. 4 shows an image demonstrating E3.5 mouse embryos in contact with the three-dimensional system during in vitro culture.

FIG. 5 shows an image demonstrating E3.5 mouse embryo developed to the E5.5 stage in the three-dimensional in vitro culture system.

FIG. 6 shows an image of a structure with an expanded body attached to the three-dimensional in vitro culture system through a stem and placenta-like structure.

FIG. 7 shows an image of a pre-implantation E4.5 mouse embryo.

FIGS. 8A-8E show images of the progress of development of an E4.5 mouse embryo in a three-dimensional in vitro culture system. FIG. 8A shows an image at day 1; FIG. 8B shows an image at day 2; FIG. 8C shows an image at day 3; FIG. 8D shows an image at day 4; and FIG. 8E shows an image at day 5.

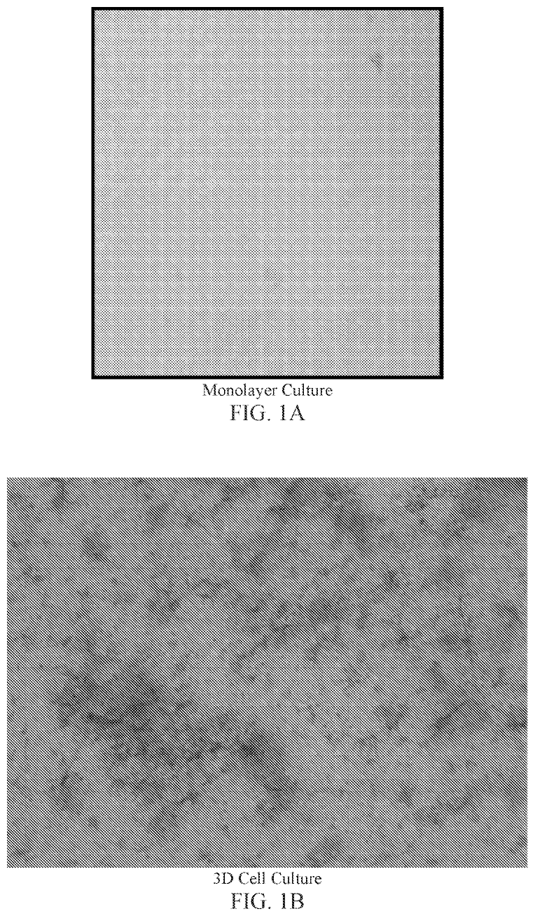

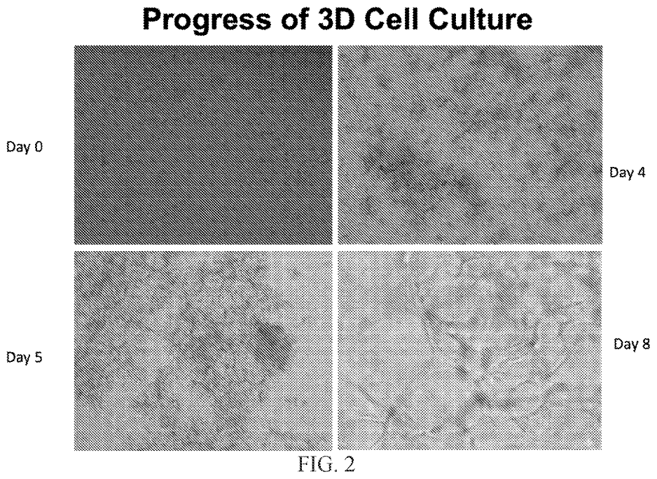

### DETAILED DESCRIPTION OF THE INVENTION

Various publications, articles and patents are cited or described in the background and throughout the specification: each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present). A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

The words “right,” “left,” “lower,” and “upper” designate directions in the drawings to which reference is made.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

### Three-Dimensional Co-Culture System

Provided herein are three-dimensional co-culture systems for culturing an embryo in vitro. The three-dimensional co-culture systems provide an in vitro culture environment closer to the in vivo environment than the flat monolayer culture system. Three-dimensional co-culture systems also provide the embryos with a three-dimensional support during their development in vitro. The three-dimensional co-culture systems and methods of using the three-dimensional co-culture systems provide for an environment that results in better embryo viability and development. Better embryo viability and development can, for example, be demonstrated by higher success rate of human in vitro fertilization (IVF), by better efficiency in reproductive technologies in agriculture-important animals, and by better efficiency for conservation of extinct and endangered species through reproductive technology.

In one general aspect, the invention relates to a three-dimensional co-culture system. The three-dimensional co-culture system can, for example, comprise (a) a three-dimensional structure: (b) at least one somatic cell; (c) at least one embryo; and (d) cell culture media.

As used herein, the term “three-dimensional co-culture” is generally understood by the person skilled in the art and refers to a method of culturing at least one type of cell with at least one embryo, wherein the at least one type of cell and the at least one embryo are implanted or seeded into or near an artificial structure (i.e., a three-dimensional structure) capable of supporting the three-dimensional co-culture of the at least one type of cell and the at least one embryo. The three-dimensional structures are critical to mimicking the in vivo milieu to allow the at least one cell and the at least one embryo to grow and/or progress in their own micro-environments. In certain embodiments, the three-dimensional co-culture systems are suitable for use in in vitro cell culture.

In certain embodiments, the three-dimensional structure is a scaffold-based structure. The scaffold-based structure can, for example, comprise a gel-like material or a structural scaffold. The gel-like material can, for example, be selected from a hydrogel, an agarose, a basement membrane extract, or an extracellular matrix. The extracellular matrix can, for example, be synthetic or natural. By synthetic, it is meant that the extracellular matrix is created under laboratory conditions. By natural, it is meant that the extracellular matrix is derived and/or isolated from a particular organism. Gel-like materials are known in the art, and are available commercially, see, e.g., MyoGel (pharmasana.co.uk); OBAGEL® (Obatala; New Orleans, LA). CETUREGEL™ (Yeasen Biotechnology: Shanghai, China), JELLAGEL® (Jellagen; Wales), GROWDEX® (UPM Biomedicals; Helsinki, Finland), GELTREX™ (Thermo Fisher; Waltham, MA), and MATRIGEL® (Corning Life Sciences; Corning, NY).

In certain embodiments, the scaffold-based structure can be created by a three-dimensional printer.

As used herein, the term “scaffold” refers to a structure, comprising a biocompatible material, that provides a surface suitable for adherence and proliferation of the at least one somatic cell and the at least one embryo. A scaffold may provide mechanical stability and support. A scaffold may be a particular shape or form so as to influence or delimit a three-dimensional shape or form assumed by the population of the at least one somatic cell and the at least one embryo. Such shapes or forms include, but are not limited to, a film (e.g., a form with two-dimensions substantially greater than the third dimension), a ribbon, a cord, a sheet, a flat disc, a cylinder, a sphere, or a three-dimensional amorphous shape.

In certain embodiments, the three-dimensional structure is a scaffold-free structure. The scaffold-free structure can, for example, comprise cell aggregates forming a three-dimensional structure.

As used herein. “cell aggregate” or “cell aggregation” refers to the clustering together and adhesion of initially separate cells to form an aggregate. A cell aggregate is one of several main types of cell organization that comprises cells that are loosely grouped together, not tightly joined, and thus, not forming tissues. Examples can include the clustering of unicellular organisms or blood cells in suspension and the condensation of mesenchymal cells during cartilage formation.

In certain embodiments, the somatic cell is selected from a skin cell, a bone cell, a blood cell, a connective tissue cell, a cumulus cell, a granulosa cell, and/or a cell from the reproductive tract (e.g., a cell from the oviduct or a cell from the uterus). The somatic cell can, for example, be from the same or from a different species as the embryo.

As used herein, “somatic cell” refers to any cell of a living organism other than a reproductive cell (i.e., the sperm and egg cells).

In certain embodiments, the embryo is selected from the group consisting of a mammalian embryo, an avian embryo, a reptilian embryo, a fish embryo, an amphibian embryo, and a marsupial embryo. The embryo can, for example, be a mammalian embryo.

In certain embodiments, the cell culture media is selected from at least one of minimum essential media (MEM), Dulbecco's modified eagle medium (DMEM), Roswell Park Memorial Institute medium (RPMI), Dulbecco's modified eagle medium/nutrient mixture F-12 (DMEM/F-12), N2B27, M16, potassium supplemented SOM (KSOM), tissue culture medium-199 (TCM-199), or a custom-made medium. The cell culture media can, for example, comprise at least one of a sodium ion (Na⁺), a potassium ion (K⁺), a calcium ion (Ca²⁺), or a magnesium ion (Mg²⁺).

In certain embodiments, the somatic cell is embedded in or placed near the three-dimensional structure. In certain embodiments, the embryo is embedded in or placed near the three-dimensional structure. The somatic cell and/or the embryo can be embedded in the three-dimensional structure or placed into a hole/cavity created within the three-dimensional structure.

In certain embodiments, the three-dimensional co-culture system further comprises a bioreactor. Bioreactors can enable the precise and reproducible control over environmental conditions for the embryo and somatic cell culture. These environmental conditions can, for example, include temperature, pH, medium flow rate, oxygen, nutrient supply, and waste metabolite removal. Additionally, increasingly complex systems are being designed for the simultaneous control of seeding cells into scaffolds. Common to these advanced systems is the ability to maintain and monitor the environment during growth.

Several designs of bioreactors exist, which include, but are not limited to, rotating wall vessels, direct perfusion systems, hollow fibers, spinner flasks, and mechanical force systems. Bioreactors are known in the art, see, e.g., Martin et al., “The role of bioreactors in tissue engineering,” Trends Biotechnol. 22:80-86 (2004).

### Methods of Use

In another general aspect, the invention relates to a method of growing an embryo in a three-dimensional co-culture system. The methods comprise (a) culturing at least one somatic cell; (b) embedding the at least one somatic cell in a three-dimensional structure; (c) obtaining at least one embryo; and (d) embedding or placing the at least one embryo in or near the three-dimensional structure; wherein the at least one somatic cell and at least one embryo are grown in or near the three-dimensional structure in cell culture media.

The embryo can, for example, be embedded in the three-dimensional structure or placed into a hole/cavity created within the three-dimensional structure. The embryo can also be placed near the three-dimensional structure. After the embryo is placed in the three-dimensional co-culture system, the culture media is changed or modified for the best development of the embryo. By way of a non-limiting example, the culture media can be changed or modified for the best development of the embryo if the requirement for the embryo is different from the somatic cell, e.g., a bovine embryo can be cultured in chemically defined culture media, while the somatic cell may prefer media supplemented with fetal bovine serum.

In certain embodiments in the methods of the invention, the three-dimensional structure is a scaffold-based structure. The scaffold-based structure can, for example, comprise a gel-like material or a structural scaffold. The gel-like material can, for example, be selected from a hydrogel, an agarose, a basement membrane extract, or an extracellular matrix. Gel-like materials are known in the art, and are available commercially, see, e.g., MyoGel (pharmasana.co.uk); OBAGEL® (Obatala; New Orleans, LA), CETUREGEL™ (Yeasen Biotechnology; Shanghai, China), JELLAGEL® (Jellagen; Wales), GROWDEX® (UPM Biomedicals; Helsinki, Finland), GELTREX™ (Thermo Fisher; Waltham, MA), and MATRIGEL® (Corning Life Sciences; Corning, NY).

In certain embodiments in the methods of the invention, the scaffold-based structure is created by a three-dimensional printer.

In certain embodiments in the methods of the invention, the three-dimensional structure is a scaffold-free structure. The scaffold-free structure can, for example, comprise cell aggregates forming a three-dimensional structure.

In certain embodiments in the methods of the invention, the somatic cell is selected from a skin cell, a bone cell, a blood cell, a connective tissue cell, a cumulus cell, a granulosa cell, and/or a cell from the reproductive tract (e.g., a cell from the oviduct or a cell from the uterus). The somatic cell can, for example, be from the same or from a different species as the embryo.

In certain embodiments, the embryo is selected from the group consisting of a mammalian embryo, an avian embryo, a reptilian embryo, a fish embryo, an amphibian embryo, and a marsupial embryo. The embryo can, for example, be a mammalian embryo.

In certain embodiments in methods of the invention, the cell culture media is selected from at least one of minimum essential media (MEM), Dulbecco's modified eagle medium (DMEM), Roswell Park Memorial Institute medium (RPMI), Dulbecco's modified eagle medium/nutrient mixture F-12 (DMEM/F-12), N2B27, M16, potassium supplemented SOM (KSOM), tissue culture medium-199 (TCM-199), or a custom-made medium. The cell culture media can, for example, comprise at least one of a sodium ion (Na⁺), a potassium ion (K⁺), a calcium ion (Ca²⁺), or a magnesium ion (Mg²⁺).

In certain embodiments in methods of the invention, the somatic cell is embedded in or placed near the three-dimensional structure. In certain embodiments, the embryo is embedded in or placed near the three-dimensional structure.

In certain embodiments in methods of the invention, the three-dimensional co-culture system further comprises a bioreactor.

### Kits

In another general aspect, the invention relates to kits comprising the components necessary for a three-dimensional co-culture system. The kits can, for example, comprise (a) a three-dimensional structure; (b) at least one somatic cell; (c) at least one embryo; and (d) cell culture media.

In certain embodiments in kits of the invention, the three-dimensional structure is a scaffold-based structure. The scaffold-based structure can, for example, comprise a gel-like material or a structural scaffold. The gel-like material can, for example, be selected from a hydrogel, an agarose, a basement membrane extract, or an extracellular matrix. Gel-like materials are known in the art, and are available commercially, see. e.g., MyoGel (pharmasana.co.uk); OBAGEL® (Obatala; New Orleans, LA), CETUREGEL™ (Yeasen Biotechnology; Shanghai, China), JELLAGEL® (Jellagen; Wales), GROWDEX® (UPM Biomedicals; Helsinki, Finland), GELTREX™ (Thermo Fisher; Waltham, MA), and MATRIGEL® (Corning Life Sciences; Corning, NY).

In certain embodiments in kits of the invention, the scaffold-based structure is created by a three-dimensional printer.

In certain embodiments in kits of the invention, the three-dimensional structure in the kit is a scaffold-free structure. The scaffold-free structure can, for example, comprise cell aggregates forming a three-dimensional structure.

In certain embodiments in kits of the invention, the somatic cell in the kit is selected from a skin cell, a bone cell, a blood cell, a connective tissue cell, a cumulus cell, a granulosa cell, and/or a cell from the reproductive tract (e.g., a cell from the oviduct or a cell from the uterus). The somatic cell can, for example, be from the same or from a different species as the embryo.

In certain embodiments, the embryo is selected from the group consisting of a mammalian embryo, an avian embryo, a reptilian embryo, a fish embryo, an amphibian embryo, and a marsupial embryo. The embryo can, for example, be a mammalian embryo.

In certain embodiments in kits of the invention, the cell culture media is selected from at least one of minimum essential media (MEM), Dulbecco's modified eagle medium (DMEM), Roswell Park Memorial Institute medium (RPMI), Dulbecco's modified eagle medium/nutrient mixture F-12 (DMEM/F-12), N2B27, M16, potassium supplemented SOM (KSOM), tissue culture medium-199 (TCM-199), or a custom-made medium. The cell culture media can, for example, comprise at least one of a sodium ion (Na⁺), a potassium ion (K⁺), a calcium ion (Ca²⁺), or a magnesium ion (Mg²⁺).

In certain embodiments in kits of the invention, the somatic cell in the kit is embedded in or placed near the three-dimensional structure. In certain embodiments, the embryo in the kit is embedded in or placed near the three-dimensional structure.

In certain embodiments, the kit further comprises a bioreactor.

### EMBODIMENTS

The invention provides also the following non-limiting embodiments.

Embodiment 1 is a three-dimensional co-culture system comprising:
(a) a three-dimensional structure;
(b) at least one somatic cell;
(c) at least one embryo; and
(d) cell culture media.

Embodiment 2 is the three-dimensional co-culture system of embodiment 1, wherein the three-dimensional structure is a scaffold-based structure.

Embodiment 3 is the three-dimensional co-culture system of embodiment 2, wherein the scaffold-based structure comprises a gel-like material or structural scaffold.

Embodiment 4 is the three-dimensional co-culture system of embodiment 3, wherein the gel-like material is selected from a hydrogel, an agarose, a basement membrane extract, or an extracellular matrix.

Embodiment 5 is the three-dimensional co-culture system of embodiment 3, wherein the structural scaffold is created by a three-dimensional printer.

Embodiment 6 is the three-dimensional co-culture system of embodiment 1, wherein the three-dimensional structure is a scaffold-free structure.

Embodiment 7 is the three-dimensional co-culture system of embodiment 6, wherein the scaffold-free structure comprises cell aggregates forming a three-dimensional structure.

Embodiment 8 is the three-dimensional co-culture system of any one of embodiments 1-7, wherein the somatic cell is selected from a skin cell, a bone cell, a blood cell, a connective tissue cell, a cumulus cell, a granulosa cell, and/or a cell from the reproductive tract.

Embodiment 9 is the three-dimensional co-culture system of any one of embodiments 1-8, wherein the embryo is selected from the group consisting of a mammalian embryo, an avian embryo, a reptilian embryo, a fish embryo, an amphibian embryo, and a marsupial embryo.

Embodiment 10 is the three-dimensional co-culture system of embodiment 9, wherein the embryo is a mammalian embryo.

Embodiment 11 is the three-dimensional co-culture system of any one of embodiments 1-10, wherein the cell culture media is selected from at least one of MEM, DMEM, RPMI, DMEM/F-12, N2B27, M16, KSOM, TCM-199, or a custom-made medium.

Embodiment 12 is the three-dimensional co-culture system of any one of embodiments 1-11, wherein the cell culture media comprises at least one of a sodium ion (Na⁺), a potassium ion (K⁺), a calcium ion (Ca²⁺), or a magnesium ion (Mg²⁺).

Embodiment 13 is the three-dimensional co-culture system of any one of embodiments 1-12, wherein the somatic cell is embedded in or placed near the three-dimensional structure.

Embodiment 14 is the three-dimensional co-culture system of any one of embodiments 1-13, wherein the embryo is embedded in or placed near the three-dimensional structure.

Embodiment 15 is the three-dimensional co-culture system of any one of embodiments 1-14, wherein the three-dimensional co-culture system further comprising a bioreactor.

Embodiment 16 is a method of growing an embryo in a three-dimensional co-culture system, the method comprising:
(a) culturing at least one somatic cell;
(b) embedding the at least one somatic cell in a three-dimensional structure;
(c) obtaining at least one embryo; and
(d) embedding or placing the at least one embryo in or near the three-dimensional structure;
   wherein the at least one somatic cell and at least one embryo are grown in or near the three-dimensional structure in cell culture media.

Embodiment 17 is the method of embodiment 16, wherein the three-dimensional structure is a scaffold-based structure.

Embodiment 18 is the method of embodiment 17, wherein the scaffold-based structure comprises a gel-like material or structural scaffold.

Embodiment 19 is the method of embodiment 18, wherein the gel-like material is selected from a hydrogel, an agarose, a basement membrane extract, or an extracellular matrix.

Embodiment 20 is the method of embodiment 18, wherein the structural scaffold is created by a three-dimensional printer.

Embodiment 21 is the method of embodiment 16, wherein the three-dimensional structure is a scaffold-free structure.

Embodiment 22 is the method of embodiment 21, wherein the scaffold-free structure comprises cell aggregates forming a three-dimensional structure.

Embodiment 23 is the method of any one of embodiments 16-22, wherein the somatic cell is selected from a skin cell, a bone cell, a blood cell, a connective tissue cell, a cumulus cell, a granulosa cell, and/or a cell from the reproductive tract.

Embodiment 24 is the method of any one of embodiments 16-23, wherein the embryo is selected from the group consisting of a mammalian embryo, an avian embryo, a reptilian embryo, a fish embryo, an amphibian embryo, and a marsupial embryo.

Embodiment 25 is the method of embodiment 24, wherein the embryo is a mammalian embryo.

Embodiment 26 is the method of any one of embodiments 16-25, wherein the cell culture media is selected from at least one of MEM, DMEM, RPMI, DMEM/F-12, N2B27, M16, KSOM, TCM-199, or a custom-made medium.

Embodiment 27 is the method of any one of embodiments 16-26, wherein the cell culture media comprises at least one of a sodium ion (Na⁺), a potassium ion (K⁺), a calcium ion (Ca²⁺), or a magnesium ion (Mg²⁺).

Embodiment 28 is a kit comprising:
(a) a three-dimensional structure;
(b) at least one somatic cell;
(c) at least one embryo; and
(d) cell culture media.

Embodiment 29 is the kit of embodiment 28, wherein the three-dimensional structure is a scaffold-based structure.

Embodiment 30 is the kit of embodiment 29, wherein the scaffold-based structure comprises a gel-like material or structural scaffold.

Embodiment 31 is the kit of embodiment 30, wherein the gel-like material is selected from a hydrogel, an agarose, a basement membrane extract, or an extracellular matrix.

Embodiment 32 is the kit of embodiment 30, wherein the structural scaffold is created by a three-dimensional printer.

Embodiment 33 is the kit of embodiment 28, wherein the three-dimensional structure is a scaffold-free structure.

Embodiment 34 is the kit of embodiment 33, wherein the scaffold-free structure comprises cell aggregates forming a three-dimensional structure.

Embodiment 35 is the kit of any one of embodiments 28-34, wherein the somatic cell is selected from a skin cell, a bone cell, a blood cell, a connective tissue cell, a cumulus cell, a granulosa cell, and/or a cell from the reproductive tract.

Embodiment 36 is the kit of any one of embodiments 28-35, wherein the embryo is selected from the group consisting of a mammalian embryo, an avian embryo, a reptilian embryo, a fish embryo, an amphibian embryo, and a marsupial embryo.

Embodiment 37 is the kit of embodiment 36, wherein the embryo is a mammalian embryo.

Embodiment 38 is the kit of any one of embodiments 28-37, wherein the cell culture media is selected from at least one of MEM, DMEM, RPMI, DMEM/F-12, N2B27, M16, KSOM, TCM-199, or a custom-made medium.

Embodiment 39 is the kit of any one of embodiments 28-38, wherein the cell culture media comprises at least one of a sodium ion (Na⁺), a potassium ion (K⁺), a calcium ion (Ca²⁺), or a magnesium ion (Mg²⁺).

Embodiment 40 is the kit of any one of embodiments 28-39, wherein the somatic cell is embedded in or placed near the three-dimensional structure.

Embodiment 41 is the kit of any one of embodiments 28-40, wherein the embryo is embedded in or placed near the three-dimensional structure.

Embodiment 42 is the kit of any one of embodiments 28-41, wherein the three-dimensional co-culture system further comprising a bioreactor.

### EXAMPLES

### Example 1: Three-Dimensional Co-Culture System

Preparation of three-dimensional structure (3-D structure): GELTREX™ (Thermo Fisher Scientific: Waltham, MA) was thawed on ice in a refrigerator at 4° C. overnight. A 1000 μl tip rack, a 100 μl tip rack, and 10-1.5 ml tubes were also placed in the refrigerator at 4° C. overnight. After incubation overnight, the thawed GELTREX™ was removed from the refrigerator and placed on ice.

Preparation of somatic cells for three-dimensional co-culture: Somatic cells (e.g., cumulus cells) were cultured in cell culture medium. The cumulus cells were washed with PBS twice. 4 drops of trypsin were added to the cell culture flask. The cells were incubated for 5-10 minutes at 38.5° C. The flask was tapped gently to detach the cells. The cells were divided into 2-1.5 ml tubes. The cells were centrifuged at 500 g for 5 minutes. The supernatant was discarded after centrifugation.

Plating the somatic cells and creating three-dimensional structure: 1 ml of regular DMEM medium was added to one of the tubes, and the cells were mixed gently. 500 μl of cells were plated into 2 wells of a 4-well plate. The 4-well plate was placed in the incubator. This preparation of cells was grown as a monolayer and was used as a control.

200 μl of GELTREX™ was added to the other tube, and the contents of the tube were mixed gently by pipetting up and down 3 times. 50 μl of the cells with the GELTREX™ were plated onto each well of a 4-well plate. The 4-well plate was placed in the incubator for 20-30 minutes. The 4-well plate was taken out of the incubator to observe the solidification of the GELTREX™. Upon solidification of the GELTREX™, 450 μl of regular DMEM was added into each well of the 4-well plate. The 4-well plate was placed in the incubator to allow formation of the three-dimensional cell culture.

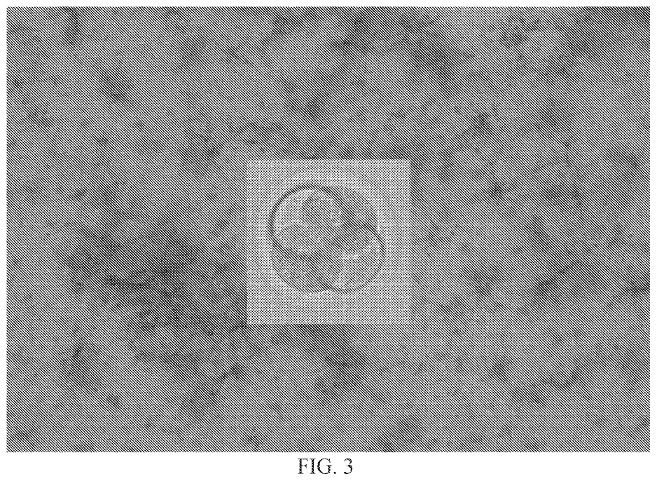

Continuing three-dimensional culture: The three-dimensional culture was checked every day under an inverted microscope to observe the cell proliferation and the formation of cell aggregates. The cell culture medium was changed as needed as indicated when the color of the media turned yellow, demonstrating a lowered pH of the cell culture medium. Photos of the three-dimensional culture were taken with a EVOS microscope (Thermo Fisher Scientific; Waltham, MA) (FIG. 2).

Embryos were either embedded within the three-dimensional co-culture on the first day of plating, or the embryos were placed into holes/cavities within the three-dimensional co-culture structure. After placement of the embryos, the three-dimensional co-culture was checked daily for cell proliferation, structural integrity, and morphology of the three-dimensional structure, and the culture medium was changed as needed for the best development of the embryos. The culture medium was changed when the color of the medium turned yellow, indicating a lowered pH for the medium. Photos of the three-dimensional co-culture were taken and the embryo development was recorded (FIG. 3).

### Example 2: Three-Dimensional Co-Culture System for Culturing Embryos

Preparation of three-dimensional structure with bovine cumulus cells: GELTREX® solution is thawed overnight at 4° C. The next day, the thawed GELTREX® solution was placed in a pre-cooled 0.5 ml tube. The amount of GELTREX® can vary based on the experiment and ranges from 200 to 600 μl.

A 10 μl single cell suspension of bovine cumulus cells was added per 100 μl of GELTREX® solution. The number of bovine cumulus cells can vary from 1,000 to 10,000 per 100 μl of GELTREX® solution. The cells were mixed with the GELTREX® solution by slowly pipetting up and down several times.

The GELTREX® and cell mixture was plated onto a 48-well plate. 50-100 μl of the mixture were seeded in each well of the 48-well plate.

The plate was incubated at 37° C. for 10 minutes to allow for the GELTREX® solution to solidify. At the end of the incubation, fresh TCM-199 medium supplemented with 15% fetal bovine serum (FBS) was added to each well. The plate was then placed in an incubator at 37° C. in an atmosphere of 5% CO₂ in air until use.

### Collection and Culture of Pre-Implantation Mouse Embryos

3-week old CD-1 female mice (Charles River Laboratories, Wilmington, MA) were treated with 5 I.U. (International Units) PMSG (pregnant mare serum gonadotropin) (BioVendor R&D, Czech Republic) and followed by 5 I.U. hCG (human chorionic gonadotropin) (Millipore; Burlington, MA) 48 hours later. The females were then paired with stud B6D2F1 or 129SV (Jackson Laboratory; Bar Harbor. ME) males overnight. The females were checked for mating plugs the next morning and only plugged females were used for embryo collection. The pre-implantation mouse embryos were collected from the plugged females.

The mouse embryos were placed in the three-dimensional GELTREX® and cell solutions in the 48-well plate prepared as described above. The mouse embryos were cultured at 37° C. in an atmosphere of 5% CO₂ in air.

The culture medium was changed every other day or as often as needed. The development of the mouse embryos was monitored daily.

### Results

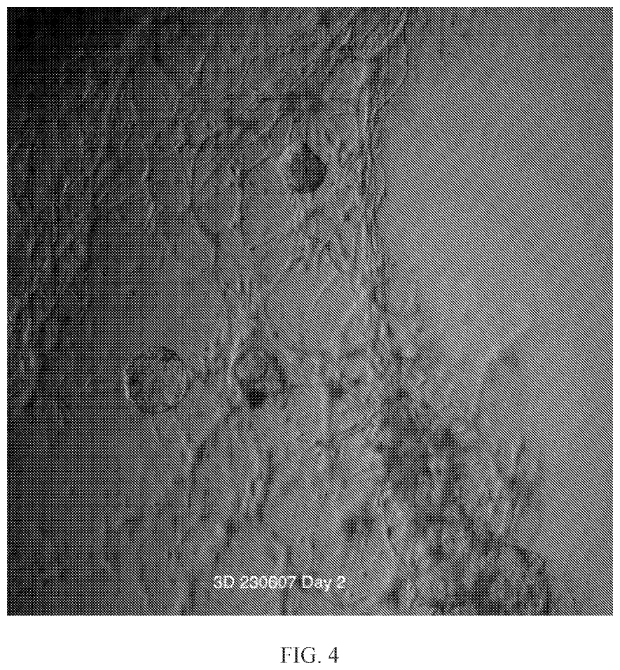

Development of pre-implantation E3.5 mouse embryos cultured in the three-dimensional in vitro culture system: Eleven (11) E3.5 mouse embryos (CD-1 female x B6DF1 male) were placed into the three-dimensional GELTREX® and bovine cumulus cell culture system. The mouse embryos were cultured in TCM-199 medium supplemented with 15% FBS at 37° C. in an atmosphere of 5% CO₂ in air. All of the E3.5 mouse embryos hatched and touched down to the three-dimensional system on the second day during culturing (FIG. 4).

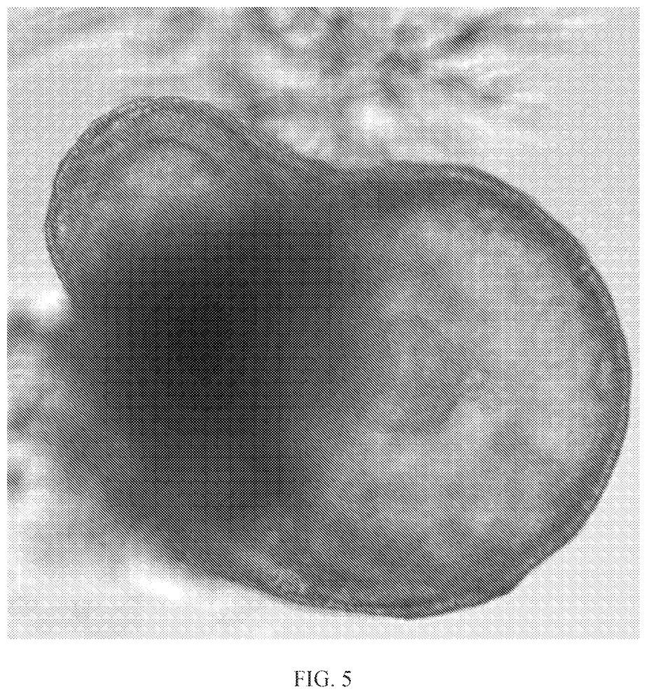

The mouse embryos continued their development and formed embryos equaling to E5.5 of their in vivo counterparts in the three-dimensional culture system (FIG. 5).

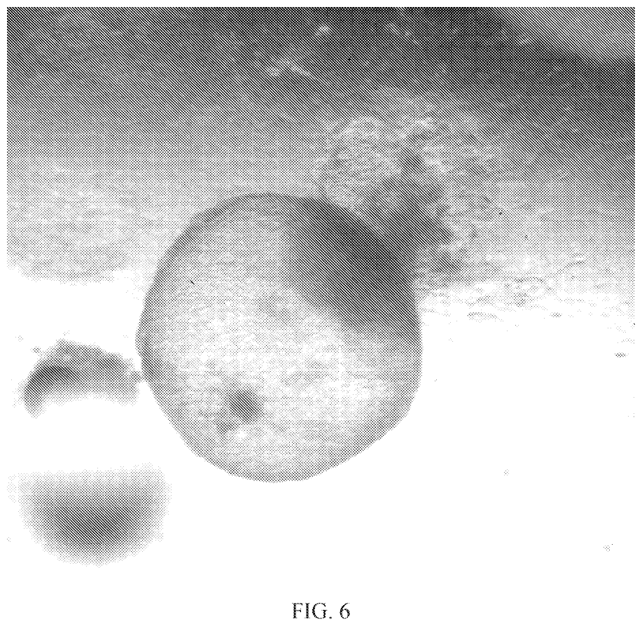

The mouse embryos continued their development in the three-dimensional culture system and six (6) of the embryos developed a structure with an expanded body attached to the three-dimensional system through a stem and a placenta-like structure (FIG. 6). The structures remained alive up to day 9 of culture.

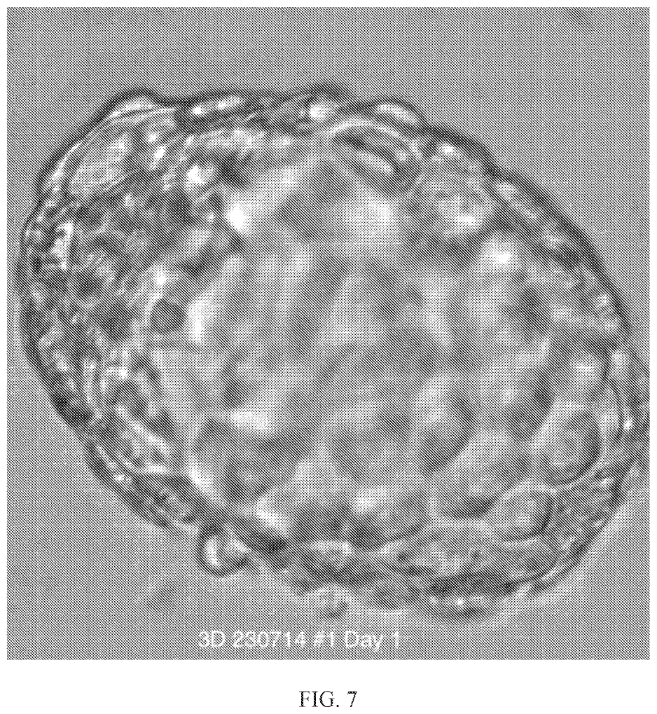

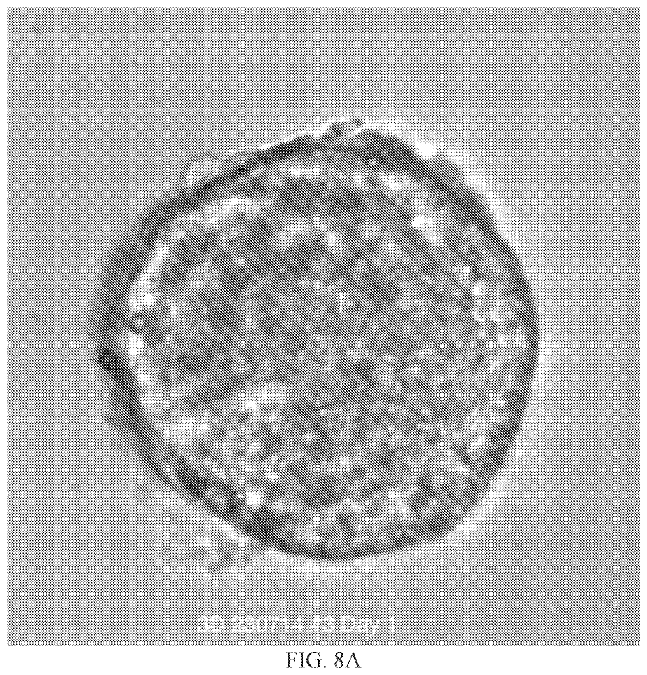

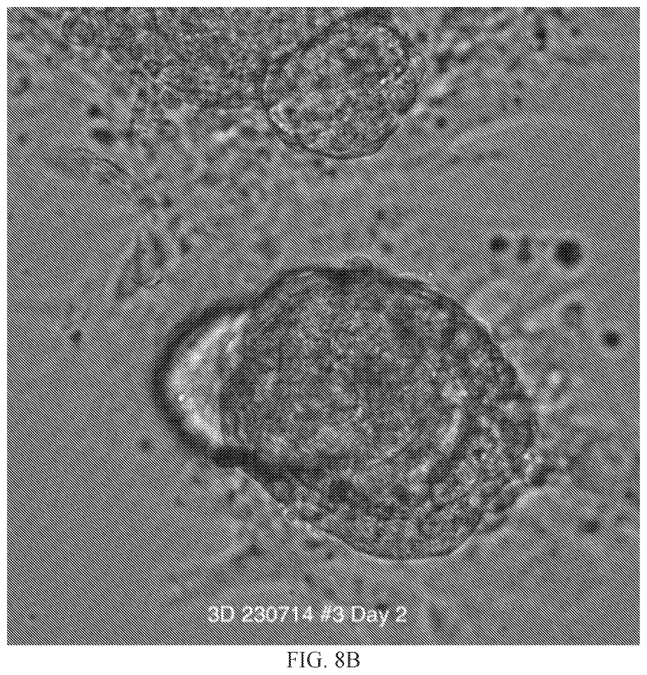

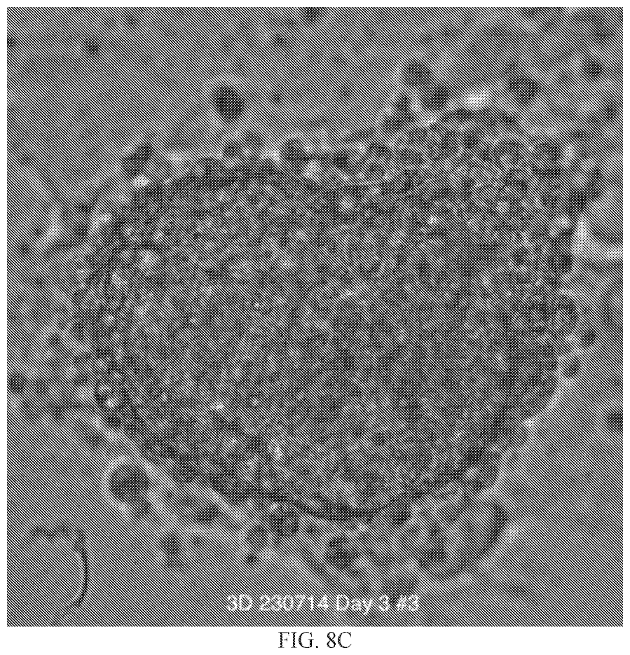

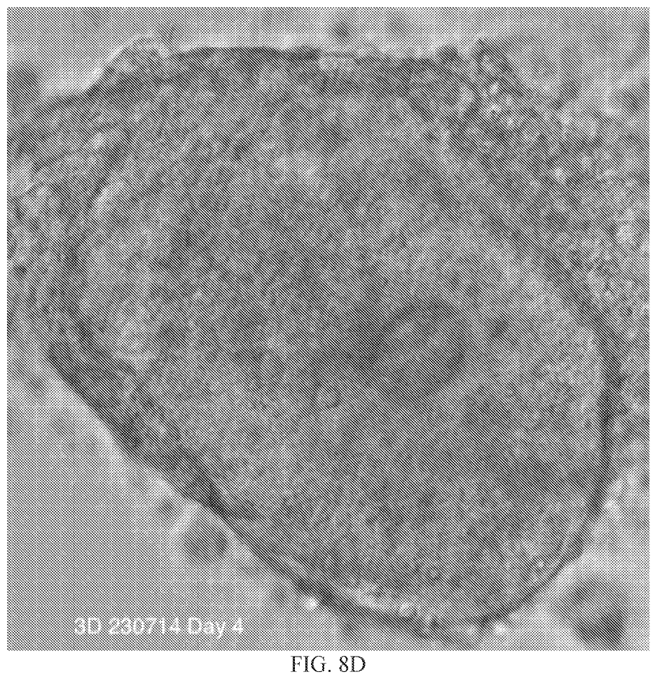

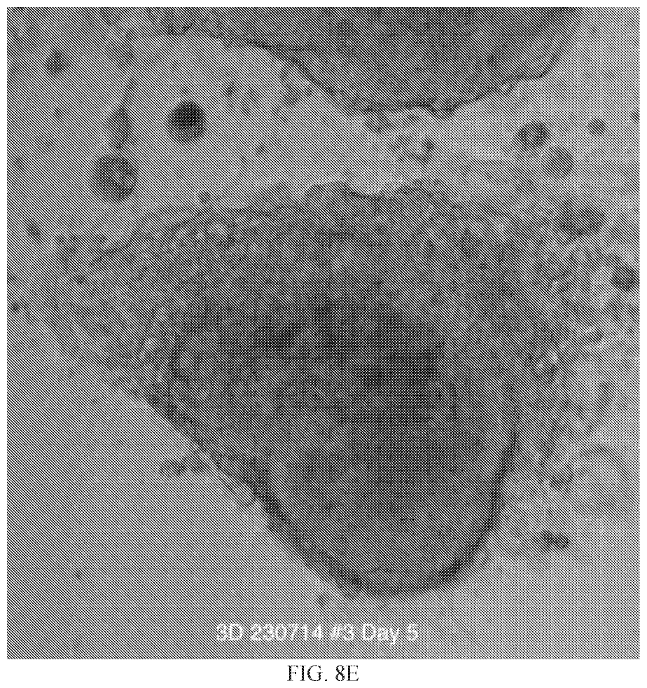

Development of rhythmic contraction of E4.5 mouse embryos cultured in the three-dimensional in vitro culture system: Pre-implantation E4.5 mouse embryos (FIG. 7) were collected from plugged CD-1 females mated with B6DF1 males and placed into the three-dimensional in vitro culture system. The mouse embryos were cultured in TCM-199 XEP medium (Table 1) at 37° C. in an atmosphere of 5% CO2 in air. FIGS. 8A-8E illustrated the progress of development of an E4.5 mouse embryo. The embryo touched down onto the three-dimensional system on the first day of culture, developed into an E5.5 embryo on the second day, and continued its development within the three-dimensional culture on Days 3, 4, and 5.

TABLE 1
TCM-199 XEP Medium

| Component | Volume | |
|---|---|---|
| TCM-199 + 15% FBS | 9.8 | mL |
| ITS-X stock, 100x | 100 | μL |
| N-acetyl-L-cysteine, 100x | 100 | μL |
| Estradiol, 1000x | 10 | μL |
| Progesterone, 1000x | 10 | μL |
| Total | 10 | mL |

On day 5 of the culture, the embryo was subjected to a treatment of 0.5 μM retinoic acid. On day 8 of the culture, rhythmic contractions resembling cardiomyocytes were noticed on the embryos. The rhythmic contractions continued to Day 18 of the culture when it slowed down and eventually stopped likely due to nutrient exhaustion. Rhythmic contractions were observed in another embryo developed in the same culture system.

### CONCLUSIONS

A static three-dimensional in vitro embryo culture system was developed and it was demonstrated that the three-dimensional in vitro culture system was capable of supporting development of pre-implantation mouse embryos beyond the post-implantation stages. The three-dimensional system is also capable of supporting organogenesis evident by the rhythmic contractions of the developing embryos. This is the first static in vitro culture system that is capable of supporting post-implantation embryo development and organogenesis in vitro.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A three-dimensional co-culture system comprising:
(a) a three-dimensional structure;
(b) at least one somatic cell;
(c) at least one embryo; and
(d) cell culture media.

2. The three-dimensional co-culture system of claim 1, wherein the three-dimensional structure is a scaffold-based structure or a scaffold-free structure.

3. The three-dimensional co-culture system of claim 2, wherein the scaffold-based structure comprises a gel-like material or structural scaffold.

4. The three-dimensional co-culture system of claim 3, wherein:
the gel-like material is selected from a hydrogel, an agarose, a basement membrane extract, or an extracellular matrix; or
the structural scaffold is created by a three-dimensional printer.

5-6. (canceled)

7. The three-dimensional co-culture system of claim 2, wherein the scaffold-free structure comprises cell aggregates forming a three-dimensional structure.

8. The three-dimensional co-culture system of claim 1, wherein:
the somatic cell is selected from a skin cell, a bone cell, a blood cell, a connective tissue cell, a cumulus cell, a granulosa cell, and/or a cell from the reproductive tract; and/or
the embryo is selected from the group consisting of a mammalian embryo, an avian embryo, a reptilian embryo, a fish embryo, an amphibian embryo, and a marsupial embryo.

9. (canceled)

10. The three-dimensional co-culture system of claim 8, wherein the embryo is a mammalian embryo.

11. The three-dimensional co-culture system of claim 1, wherein the cell culture media: is selected from at least one of MEM, DMEM, RPMI, DMEM/F-12, N2B27, M16, KSOM, TCM-199, or a custom-made medium; and/or comprises at least one of a sodium ion (Na⁺), a potassium ion (K⁺), a calcium ion (Ca²⁺), or a magnesium ion (Mg²⁺).

12. (canceled)

13. The three-dimensional co-culture system of claim 1, wherein the somatic cell and/or the embryo is embedded in or placed near the three-dimensional structure.

14. (canceled)

15. The three-dimensional co-culture system of claim 1, wherein the three-dimensional co-culture system further comprises a bioreactor.

16. A method of growing an embryo in a three-dimensional co-culture system, the method comprising:
(a) culturing at least one somatic cell;
(b) embedding the at least one somatic cell in a three-dimensional structure;
(c) obtaining at least one embryo; and
(d) embedding or placing the at least one embryo in or near the three-dimensional structure;
wherein the at least one somatic cell and at least one embryo are grown in or near the three-dimensional structure in cell culture media.

17. The method of claim 16, wherein the three-dimensional structure is a scaffold-based structure or a scaffold-free structure.

18. The method of claim 17, wherein the scaffold-based structure comprises a gel-like material or structural scaffold.

19. The method of claim 18, wherein:
the gel-like material is selected from a hydrogel, an agarose, a basement membrane extract, or an extracellular matrix; and/or
the structural scaffold is created by a three-dimensional printer.

20-21. (canceled)

22. The method of claim 16, wherein the scaffold-free structure comprises cell aggregates forming a three-dimensional structure.

23. The method of claim 16, wherein:
the somatic cell is selected from a skin cell, a bone cell, a blood cell, a connective tissue cell, a cumulus cell, a granulosa cell, and/or a cell from the reproductive tract; and/or
the embryo is selected from the group consisting of a mammalian embryo, an avian embryo, a reptilian embryo, a fish embryo, an amphibian embryo, and a marsupial embryo.

24. (canceled)

25. The method of claim 23, wherein the embryo is a mammalian embryo.

26. The method of claim 16, wherein the cell culture media: is selected from at least one of MEM, DMEM, RPMI, DMEM/F-12, N2B27, M16, KSOM, TCM-199, or a custom-made medium; and/or comprises at least one of a sodium ion (Na⁺), a potassium ion (K⁺), a calcium ion (Ca²⁺), or a magnesium ion (Mg²⁺).

27. (canceled)

28. A kit comprising:
(a) a three-dimensional structure;
(b) at least one somatic cell;
(c) at least one embryo; and
(d) cell culture media.

29. The kit of claim 28, wherein the three-dimensional structure is a scaffold-based structure or a scaffold-free structure.

30. The kit of claim 29, wherein the scaffold-based structure comprises a gel-like material or structural scaffold.

31. The kit of claim 30, wherein: the gel-like material is selected from a hydrogel, an agarose, a basement membrane extract, or an extracellular matrix; and/or the structural scaffold is created by a three-dimensional printer.

32. (canceled)

34. The kit of claim 29, wherein the scaffold-free structure comprises cell aggregates forming a three-dimensional structure.

35. The kit of claim 28, wherein:
the somatic cell is selected from a skin cell, a bone cell, a blood cell, a connective tissue cell, a cumulus cell, a granulosa cell, and/or a cell from the reproductive tract; and/or
the embryo is selected from the group consisting of a mammalian embryo, an avian embryo, a reptilian embryo, a fish embryo, an amphibian embryo, and a marsupial embryo.

36. (canceled)

37. The kit of claim 35, wherein the embryo is a mammalian embryo.

38. The kit of claim 28, wherein the cell culture media: is selected from at least one of MEM, DMEM, RPMI, DMEM/F-12, N2B27, M16, KSOM, TCM-199, or a custom-made medium; and/or comprises at least one of a sodium ion (Na⁺), a potassium ion (K⁺), a calcium ion (Ca²⁺), or a magnesium ion (Mg²⁺).

39. (canceled)

40. The kit of claim 28, wherein the somatic cell is embedded in or placed near the three-dimensional structure.

41. (canceled)

42. The kit of claim 28, wherein the three-dimensional co-culture system further comprises a bioreactor.
